SEQUENCE LISTING (2 sequences)

SEQ ID NO: 1 - protein, 1268 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 2 - DNA, 18407 bp, Synthetic construct
  source 1..18407: mol_type = other DNA
ccgtgagttacgataaaaggcgaaagagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggttagaaaaaatacgggtagaagccgccaccatgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtgataaccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaaaaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgac

(19) United States
(12) Patent Application Publication
(10) Pub. No.: US 2025/0375515 A1
(43) Pub. Date: Dec. 11, 2025

(54) RECOMBINANT VACCINE AGAINST COVID-19 TO PRODUCE CELLULAR RESPONSE IN INDIVIDUALS WITH PRE-EXISTING IMMUNITY

(71) Applicants: LABORATORIO AVI-MEX, S.A. DE C.V., Mexico City (MX); CONSEJO NACIONAL DE HUMANIDADES, CIENCIAS Y TECNOLOGÍAS, Mexico City (MX); ICAHN SCHOOL OF MEDICINE AT MOUNT SINAI, New York, NY (US)

(72) Inventors: Bernardo LOZANO DUBERNARD, Mexico City (MX); Ernesto SOTO PRIANTE, Mexico City (MX); David SARFATI MIZRAHI, Mexico City (MX); Hector Elias CHAGOYA CORTES, Mexico City (MX); Constantino III Roberto LOPEZ MACIAS, Mexico City (MX); Peter PALESE, New York, NY (US); Adolfo GARCIA-SASTRE, New York, NY (US); Florian KRAMMER, New York, NY (US); Weina SUN, New York, NY (US); Martha TORRES ROJAS, New York, NY (US)

(21) Appl. No.: 18/693,472

(22) PCT Filed: Sep. 20, 2022
(86) PCT No.: PCT/IB2022/058886

§ 371 Date: Mar. 19, 2024

(30) Foreign Application Priority Data
Sep. 20, 2021 (MX) MX/A/2021/011439

(51) Int. Cl. A61K 39/215; A61K 39/00; C12N 7/00

(52) U.S. Cl. CPC A61K 39/215; C12N 7/00; A61K 2039/5256; A61K 2039/543; A61K 2039/545; C12N 2760/18121; C12N 2770/20022; C12N 2770/20034; C12N 2770/20071

(57) ABSTRACT

A recombinant vaccine is described, which comprises an active Newcastle disease viral vector (NDV) having inserted an exogenous nucleotide sequence of severe acute respiratory syndrome coronavirus 2 (SARS-COV-2), without adjuvant, capable of generating a significant cellular response in T cells (CD4+ or CD8+) when stimulated with the S protein of the SARS-CoV-2 virus or proteins derived from it in individuals with previous immunity.

### CROSS-REFERENCE TO RELATED APPLICATIONS

The present application is a National Stage entry of International Patent Application No. PCT/IB2022/058886, filed 20 Sep. 2022 and published as International Patent Application Publication No. WO 2023/042181 A1, which claims priority to, and the benefit of, Mexican Patent Application No. MX/a/2021/011439, filed 20 Sep. 2021, each of which is incorporated by reference herein in its entirety for all purposes.

### INCORPORATION BY REFERENCE

In compliance with 37 C.F.R. 1.52 (c), the sequence information contained in electronic file name: CVZ0011US Sequence listing.xml; size 22.1 KB; created on: 22 Jan. 2025, is incorporated herein by reference in its entirety.

### TECHNICAL FIELD

The present invention is related to the techniques used in the prevention and control of coronavirus disease 2019 (COVID-19), and more particularly it is related to a recombinant viral vector vaccine that has inserted an exogenous nucleotide sequence encoding proteins with antigenic activity against severe acute respiratory syndrome coronavirus 2 (SARS-COV-2) useful for producing increased cellular response in patients with pre-existing immunity.

### BACKGROUND ART

Coronaviruses (CoV) are a family of viruses that cause the common cold and serious diseases such as Middle East Respiratory Syndrome (MERS-COV) and Severe Acute Respiratory Syndrome (SARS-COV). Severe acute respiratory syndrome coronavirus 2 (SARS-COV-2) is the etiologic agent of the coronavirus disease 2019 (COVID-19) outbreak, which began in December 2019 in Wuhan, China. On Mar. 11, 2020, the World Health Organization (WHO) declared COVID-19 as a pandemic.

The unprecedented development of vaccine options against COVID-19 has given rise to different alternatives that are already available and have been approved on an emergency basis in several countries around the world. However, the ability to generate a robust cellular response in people who receive the vaccine, given the current conditions of the pandemic is still unknown. There are many people who were infected by the virus, but were asymptomatic to the disease, or people who, having been vaccinated, did not produce enough antibodies or, having produced them, they have reduced over time.

For example, among the vaccines that have been most widely studied and used worldwide are those based on mRNA technology that use the S (Spike) protein of the SARS-CoV-2 virus. As reported by Goldberg et al. (2021—Waning immunity of the BNT162b2 vaccine: A nationwide study from Israel), the rates of documented SARS-COV-2 and severe COVID-19 infections show a statistically significant increase with time from the second dose of the vaccine, finding a greater protection between 1.6 and 1.7 times in individuals vaccinated with two doses of BNT162b2 vaccine compared to those vaccinated 2 months earlier.

The above has alerted about the importance of having a product that, regardless of its ability to produce neutralizing antibodies, can generate a significant cellular response that allows individuals to respond efficiently to a virus infection, even when the humoral response is reduced with time. Regarding the same mRNA technology, the report by Sahin et al. (Nature, 2020—COVID-19 vaccine BNT162b1 elicits human antibody and T_{H}1 T cell responses) on the humoral and cellular response of the BNT162b1 vaccine at different doses, shows that at the doses at which the vaccine was authorized (30 μg), the percentages of interferon γ-producing CD8+ cells were less than 1% with a mean of 0.22%, and even for the high dose (50 μg) a mean of 1.44 was obtained, which was not tested in terms of its safety Phase I study (Mulligan et. al. (Nature, 2020—“Phase I/II study of COVID-19 RNA vaccine BNT162b1 in adults”). It should be noted that the cellular response was significantly higher than that of donors who had undergone COVID-19 with at least 14 days in advance and no longer had symptoms of the disease, which did not reach average responses greater than 0.02% nor did they reach 0.1% in any case of the same type of interferon γ-producing CD8+ cells.

The case of adenovirus-based vaccines is similar. As reported by Zhu et al. (The Lancet, 2020—“Safety, tolerability, and immunogenicity of a recombinant adenovirus type-5 vectored COVID-19 vaccine: a dose-escalation, open-label, non-randomised, first-in-human trial”), on day 28 post-vaccination, the detected percentage of interferon γ-producing CD8+ cells did not reach 1% (100), which shows a significant but lower performance in cellular response than the mRNA vaccine.

Additionally, the identification of different variants of the SARS-COV-2 virus has led the World Health Organization to identify some of them as variants of concern, understood as those for which there is evidence of greater transmissibility, more severe disease (for example, more hospitalizations or deaths), a substantial reduction in neutralization by antibodies generated during a previous infection or by vaccination, less effectiveness of treatments or vaccines, or difficulties in detection or diagnosis. In this regard, it is expected that a robust cellular response can deal with the variants thanks to the possibility of giving rise to responses that lead to the production of antibodies that identify other epitopes of the SARS-CoV-2 S protein that do not have the same variations and consequently prevent the progression of the infection.

Consequently, it has not been possible to obtain a vaccine that can provide a significant cellular response that promotes long-term protection, regardless of the short-term humoral response that the prior art vaccines may present, and that in addition, in a dose that has proven its safety, can be used to provide an increased cellular response in individuals who previously fell ill with COVID-19, had it asymptomatically, or have been vaccinated with any mRNA, recombinant vector or inactivated SARS-COV-2 virus vaccine.

### OBJECTS OF THE INVENTION

Taking into account the drawbacks of the prior art, it is an object of the present invention to provide a recombinant viral vector vaccine capable of increasing the specific cellular response against coronavirus disease 2019 (COVID-19) obtained with a complete vaccination schedule with mRNA, recombinant viral vector technologies or a circulating SARS-COV-2 virus.

It is another object of the present invention to provide a vaccine for the control of COVID-19 that promotes a significant cellular response at a dose that has been proven to be safe.

It is another object of the present invention to provide a vaccine for the control of COVID-19 that can be used in individuals who previously got sick with COVID-19, had it asymptomatically, or have been vaccinated with an mRNA, recombinant vector or inactivated SARS-COV-2 virus vaccine.

These and other objects are achieved by a recombinant vaccine against COVID-19 in paramyxovirus viral vector according to the present invention.

### SUMMARY OF THE INVENTION

A recombinant vaccine has been invented, which comprises an active (live) viral vector of Newcastle disease having inserted an exogenous nucleotide sequence of severe acute respiratory syndrome coronavirus 2 (SARS-COV-2), without adjuvant, capable of generating a significant cellular response in T cells (CD4+ or CD8+) when stimulated with the S protein of the SARS-COV-2 virus or proteins derived thereof in individuals with previous immunity.

### BRIEF DESCRIPTION OF DRAWINGS

The novel aspects that are considered characteristic of the present invention will be established with particularity in the appended claims. However, some embodiments, characteristics and some objects and advantages thereof, will be better understood in the detailed description, when read in connection with the attached drawings, in which:

FIG. 1 is a graph of the IgG-type antibody titers (−log 2×40) against the SARS-CoV-2 S protein contained in the vaccine, present in patients with COVID-19 and in people vaccinated with the Pfizer mRNA vaccine, obtained in the experiments of Example 2.

FIG. 2 is a graph of interferon γ-producing CD4+ T cells from the severely ill patients of Example 2.

FIG. 3 is a graph of interferon γ-producing CD8+ T cells from the severely ill patients of Example 2.

FIGS. 4A and 4B show, respectively, the percentage of proliferation of T cells and the percentage of production of interferon γ in CD4+ cells of the severely ill patients of Example 2.

FIGS. 5A and 5B show, respectively, the percentage of proliferation of T cells and the percentage of production of interferon γ in CD8+ cells of the severely ill patients of Example 2.

FIGS. 6A and 6B show, respectively, the percentage of T cell proliferation and the percentage of interferon γ production in CD4+ cells of individuals immunized with 2 doses of the mRNA vaccine of Example 2.

FIGS. 7A and 7B show, respectively, the percentage of T cell proliferation and the percentage of interferon γ production in CD8+ cells of individuals immunized with 2 doses of the mRNA vaccine of Example 2.

FIGS. 8A and 8B show, respectively, the percentage of T cell proliferation and the percentage of interferon γ production in CD4+ cells of individuals immunized with 2 doses of the Pfizer-BioNTech mRNA vaccine and a third booster dose with the AstraZeneca recombinant vaccine of Example 2.

FIGS. 9A and 9B show, respectively, the percentage of T cell proliferation and the percentage of interferon γ production in CD8+ cells of individuals immunized with 2 doses of the Pfizer-BioNTech mRNA vaccine and a third booster dose with the AstraZeneca recombinant vaccine of Example 2.

FIGS. 10A, 10B and 10C show the percentage of individuals who tested positive for antibodies against SARS-COV-2 S-glycoprotein in Example 3, at days 21, 28 and 42 from the first vaccination.

FIG. 11 shows the increase in the percentage of interferon γ-producing T cells from the experiments in Example 3.

### DESCRIPTION OF EMBODIMENTS

During the development of the present invention, it has been unexpectedly found that a recombinant vaccine comprising an active paramyxovirus viral vector having inserted an exogenous nucleotide sequence encoding antigenic sites of severe acute respiratory syndrome coronavirus 2 (SARS-COV-2), and a pharmaceutically acceptable vehicle and/or excipient, without adjuvant, is capable of promoting an increase in the percentage of T cells (CD4+ or CD8+) in individuals with previous immunity to SARS-COV-2, either by having been vaccinated with mRNA vaccines, with other recombinant viral vector vaccines, or with inactivated SARS-COV-2 virus vaccines, as well as by natural infection of the same virus, which can be used in a single dose.

To achieve the increased cellular response, the viral vector used must be active (live), that is, the recombinant virus that works as a viral vector and contains the nucleotide sequence encoding antigenic sites of SARS-COV-2 has the ability to replicate.

Preferably, the viral vector used is the La Sota strain of the Newcastle disease virus, which has inserted an exogenous nucleotide sequence encoding the spike protein (Spike or S) of the SARS-COV-2 virus.

In a preferred embodiment, the sequence of the S protein has at least 80% of identity with the sequence encoding the two subunits S1 and S2 of the spike S glycoprotein of SARS-COV-2 stabilized in its prefusion form by the inclusion of at least two proline substitutions in the S2 subunit, and more preferably the sequence has at least 80% of identity with the amino acid sequence of SEQ ID NO:1.

The exogenous nucleotide sequence encoding SARS-COV-2 antigenic sites of the vaccine of the present invention can be prepared by chemical synthesis of the nucleotide sequence of interest so that it can subsequently be inserted it into the NDV viral vector. Insertion of the exogenous nucleotide sequence is performed using standard cloning techniques of molecular biology and can be inserted into any of the intergenic regions of the NDV genome. The infectious clone thus produced is transfected into a cell culture for generating recombinant virus or parent virus.

The virus replicates through consecutive passages in any system suitable for growing, such as SPF chicken embryo, or commercial cell lines or expressly designed to grow viruses, until reaching the concentration of virus that is required to achieve antigenic response, preferably between 10^{6.0} and 10^{10.0} CIED_{50%} (Chicken Embryo Infectious Dose_{50%})/mL. It is preferred that the virus be stable after at least three consecutive passages in the system used for growth once rescued from cell culture, so that a stable production is achieved on an industrial scale. For virus isolation, the virus is removed from the system suitable for growth and is separated from cellular or other components, typically by well-known clarification procedures such as filtration, ultrafiltration, gradient centrifugation, ultracentrifugation, and column chromatography, and can be further purified as desired using well known procedures, e.g., plaque assays.

Pharmaceutically acceptable vehicles for the vaccines of the present invention are preferably aqueous solutions that maintain the active virus with replication capacity.

Regarding the administration of the vaccine, it has been found that the increased cellular response is achieved by the application of at least one dose with a viral titer of at least 1×10^{8.0} measured per chicken embryo infectious dose 50% (CEID_{50%}), by intramuscular and/or intranasal route.

In a preferred embodiment, the vaccine is administered at least once, by intramuscular or intranasal route, in its active form, the intranasal route being preferred, particularly when the individual has previously been immunized with any other vaccine against COVID-19 or suffered a previous infection of the same disease through the intramuscular route.

The vaccine of the present invention is applied once by the intranasal or intramuscular route after a period of at least 90 days counted from the date on which the individual received the last immunization or recovered from the COVID-19 disease.

Preferably, the vaccine of the present invention is formulated with a volume of 0.5 mL per dose that contains the virus concentration corresponding to its intramuscular application, either in its active or inactivated form. In the embodiment in which the administration route is intranasal, the preferred volume per dose is 0.2 mL.

The vaccine in accordance with the principles of the present invention, additionally, does not cause life-threatening adverse events in mammals, particularly in humans, at high doses of the antigen of at least 1×10^{8.0} CIED_{50%}, neither severe adverse events attributable to the vaccine.

The vaccines of the present invention, through the use of a Newcastle Disease virus (NDV) vector and the inserted gene of S protein, have the ability to promote the proliferation of interferon γ-producing CD8+ or CD4+ T cells, statistically significant when stimulated with the S protein of the SARS-COV-2 virus or peptides derived from it in individuals who had previous immunity to the SARS-COV-2 virus.

The present invention will be better understood from the following examples, which are presented only for illustrative purposes to allow a full understanding of the preferred embodiments of the present invention, without implying that there are no other, non-illustrated embodiments that may be implemented based on the detailed description above.

### EXAMPLES

### Example 1

Generation of Recombinant NDV LaSota Virus with Spike S1/S2 Protein SARS-COV-2/Hexapro

By means of the methods described by Sun et al. (2020, Op. Cit.), it was obtained the construction called rNDVLS/Spike S1/S2 SARS-COV-2/Hexapro with a sequence of the ectodomain of the spike S glycoprotein of SARS-COV-2 stabilized in its prefusion form and four additional prolines distributed in the synthetic gene to give greater stability to the Spike protein expressed by NDV, inserted in a recombinant Newcastle Disease virus of nucleotide sequence SEQ ID NO:2. General methods have also been previously described for example in international publication WO2010058236A1. Viruses obtained in chicken embryos as described in the prior art were purified from FAA as previously described also in the prior art (SANTRY, Lisa A., et al. Production and purification of high-titer Newcastle disease virus for use in preclinical mouse models of cancer. Molecular Therapy-Methods & Clinical Development, 2018, vol. 9, p. 181-191; and NESTOLA, Piergiuseppe, et al. Improved virus purification processes for vaccines and gene therapy. Biotechnology and bioengineering, 2015, vol. 112, no 5, p. 843-857).

Active vaccines were prepared to be administered intramuscularly and intranasally in aqueous solution under good manufacturing practices. For this, the purified FAA was mixed with a stabilizing solution (TPG) in such a way that three vaccines were obtained with four different concentrations, according to the volume required to apply the vaccine and provide a minimum of 10^{8.0} CIED50%/mL per dose (High) to be applied to healthy volunteers.

### Example 2

Response of Cells from People Infected with SARS-COV-2 and mRNA Technology Vaccine

Peripheral blood samples were taken from severely ill (C19 G) and critically ill (C-19 C) individuals in the acute phase of COVID-19, and from individuals in the convalescent phase that previously had severe or critical illness of COVID-19 (Conv G and Conv C, respectively) within the first peak of the pandemic (June-December 2020) with a positive RT-PCR test for SARS-COV-2, and serum and peripheral blood mononuclear cells (PBMCs) and plasma were obtained. Likewise, peripheral blood samples were taken from individuals immunized with 2 doses of the Pfizer-BioNTech mRNA vaccine, and from individuals immunized with 2 doses of the Pfizer-BioNTech mRNA vaccine and a third booster dose with the AstraZeneca recombinant vaccine. By means of the ELISA immunoassay, the determination of the binding of specific antibodies against the S protein of SARS-COV-2 expressed in the vaccine of Example 1 was carried out.

In order to determine whether the antibodies against SARS-COV-2 of the individuals described above recognize the virus of Example 1, the IgG type antibody titers obtained with the ELISA immunoassay were measured by fixing on the plates a Newcastle disease virus La Sota strain (NC-LS), as well as against a vectored Newcastle disease virus without exogenous gene insert (Vc-NC-LS), the virus of Example 1 (NDV-S-hexa-pro), and a positive control of the receptor binding site of the S glycoprotein (RBD), all at a final concentration of 200 ng/100 μL. Serial dilutions (1:2) were then added starting with a 1:40 dilution of the tested sera and goat anti-human IgG antibody labeled with horseradish peroxidase as second antibody. The reaction was revealed with hydrogen peroxide and orthophenylenediamine. The titers express the dilution at which the 3-fold background optical density is reached.

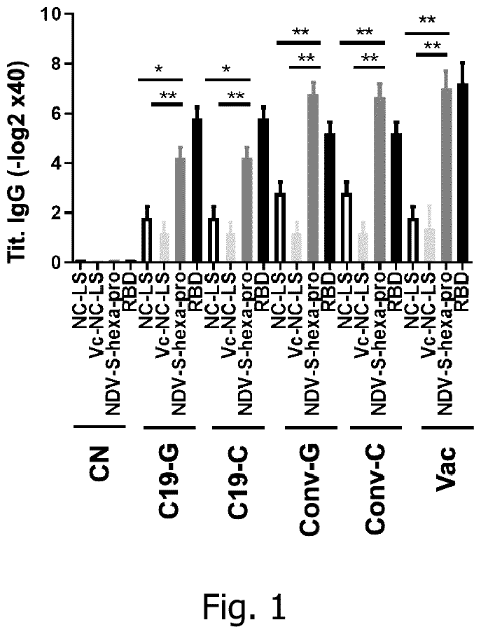

The results are shown in FIG. 1, in which it can be seen that the titers obtained with the virus of Example 1 had statistical significance (*) by the Kruskal Wallis test, p>0.05, with respect to NC-LS and to Vc-NC-LS, in all groups of subjects with previous immunity.

The results obtained show that the antibodies of individuals previously infected or immunized (with the mRNA vaccine or with the mRNA vaccine and a booster with the AstraZeneca recombinant vaccine) are capable of recognizing the viral vector used in the vaccine of the present invention and that therefore said vaccine would be capable of generating an immune response in individuals with previous immunity against the SARS-COV-2 virus.

Furthermore, in order to determine the proliferation capacity of T cells from the same individuals, the technique of stimulating T lymphocytes from their peripheral blood was performed, using a ficoll gradient and centrifugation, followed by incubation for 72 hours with 5% CO₂, to carry out subsequently the stimulation with the same viruses used for the measurement of antibodies, and a peptide activator of the S protein of SARS-COV-2 (Peptivator) as well as with phytohemagglutinin (PHA) as positive controls, to finally carry out the proliferation staining.

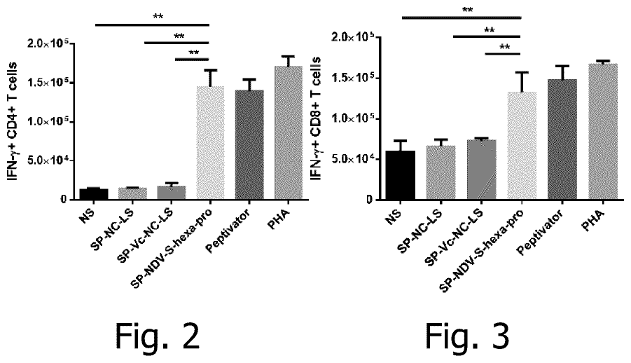

The results corresponding to severely ill patients for interferon γ-producing CD4+ and CD8+ cells are shown in FIGS. 2 and 3, respectively, where it is observed that the viral vector used in the vaccine of the present invention is capable of stimulating T lymphocytes and causing a statistically significant cellular response both in CD4+ as well as CD8+ cells. Non-significant results appear as NS.

Additionally, the percentage of proliferation of T cells and the percentage of production of interferon γ in T cells stimulated with the empty vector of Newcastle disease virus La Sota strain (Vc-NC-LS), with the vaccine of the present invention (AVX/COVID-12), and with Peptivator as positive control, were measured. The same measurements were made in non-stimulated T cells (SE).

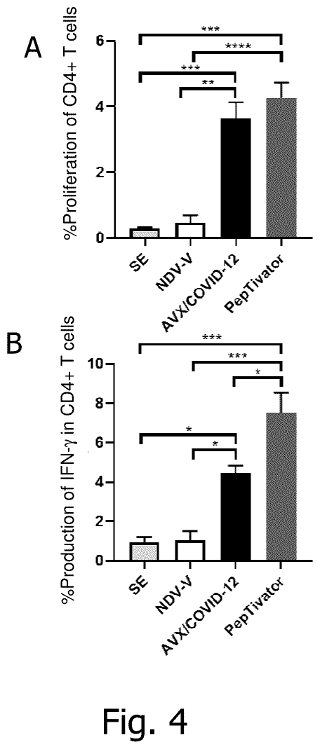

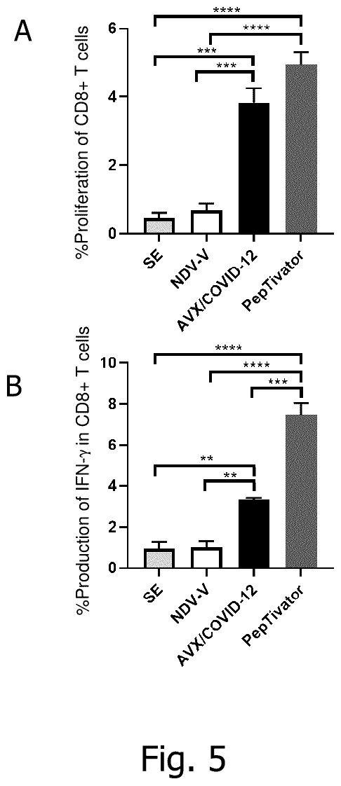

FIGS. 4A and 4B, and FIGS. 5A and 5B, show the results corresponding to patients with severe COVID-19, for interferon γ-producing CD4+ and CD8+ cells.

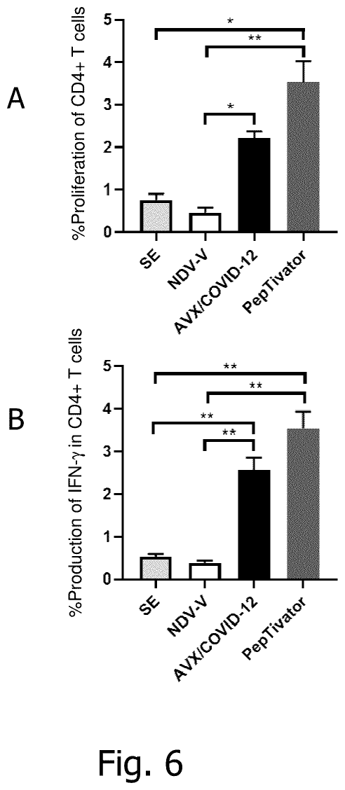

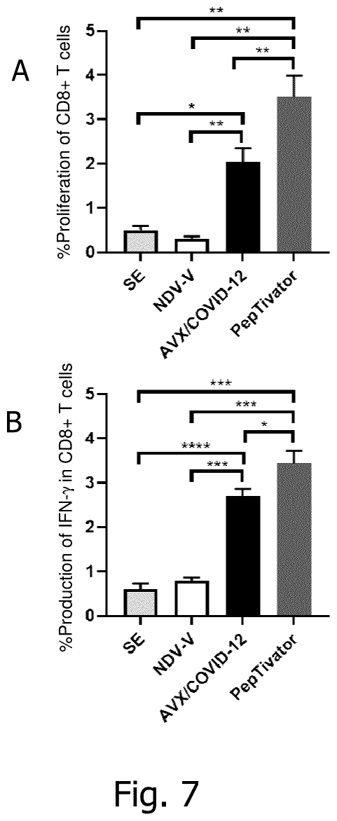

Likewise, the results corresponding to individuals immunized with 2 doses of the mRNA Pfizer-BioNTech vaccine only, for interferon γ-producing CD4+ and CD8+ cells are shown in FIGS. 6A and 6B, and in FIGS. 7A and 7B, respectively.

From these results, a greater proliferation response can be observed in the case of PBMC obtained from patients than from individuals vaccinated with the mRNA vaccine, since, during the pathology, the induced response is generally associated with effector T cells which, after the recovery of the patient, enter a refractory phase, inducing immunological memory, on the other hand, 6 months after vaccination, in the bloodstream we would only find a memory response induced by vaccine epitopes. In both cases, the response guided by effector and memory T cells are by epitopes expressed in the vaccine of the present invention, inducing effector activities, as would occur with the naive S protein and the vaccine antigen expressed by the mRNA vaccine.

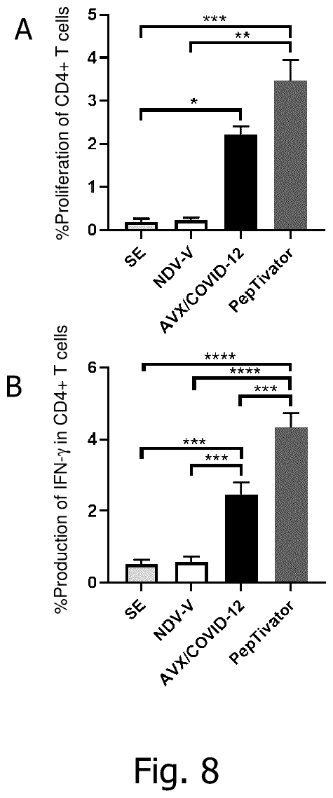

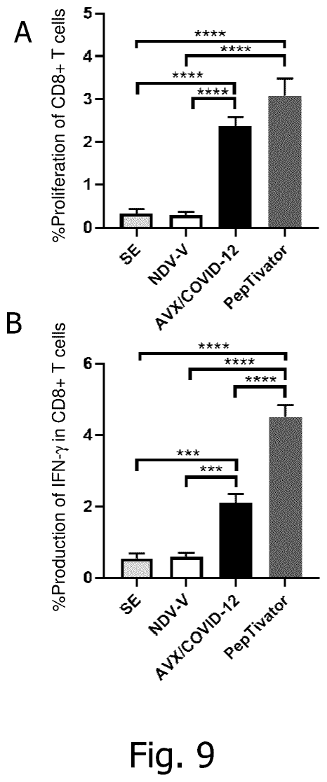

Finally, FIGS. 8A and 8B, and FIGS. 9A and 9B show, respectively, the results for interferon γ-producing CD4+ and CD8+ cells, of individuals immunized with 2 doses of the mRNA Pfizer-BioNTech vaccine and a third booster dose with the AstraZeneca recombinant vaccine. From these results, it can be observed that the cellular response is similar to those already analyzed by infection or by immunization with mRNA vaccine. However, a general trend towards proliferation and interferon γ production similar to that observed in COVID-19 patients is observed, with significant differences between induction by the empty vector and the vaccine of the present invention in CD8+ T cells (p=0.0005, p<0.0001 for proliferation and p=0.0081, p=0.0004 for IFN-γ respectively).

Consequently, it is shown that by using the viral vector of Example 1 in the vaccine of the present invention, it is possible to stimulate in vitro the cellular response in individuals with previous immunity against SARS-COV-2.

### Example 3

### Study to Evaluate the Level of Safety and Immunogenicity Produced by the Active Vaccine Against COVID-19 in Humans

A study to evaluate the safety and immunogenicity of the vaccine in accordance with the principles of the present invention in healthy volunteers was carried out, according to protocols authorized by the regulatory authorities.

For this study, the virus of example 1 applied in high doses in groups of 10 individuals was used as follows:

TABLE 1
Groups per route of administration

| Route Dose 1 (Day | Route Dose 2 (Day |
|---|---|
| 0) | 21) |
| IN | IN |
| IN | IM |
| IM | IM |

where:
IN=Intranasal, 0.2 mL
IM=Intramuscular, 0.5 mL

The second dose was applied on day 21 after the first dose, and samples were taken from the participants on the baseline day (day 0), the day of the second vaccination (day 21) prior to the second vaccination, one week after the second vaccination (day 28) and finally three weeks after the second vaccination (day 42). Neutralization tests were carried out on the blood samples of individuals immunized with each of the doses and routes, using a surrogate ELISA GenScript® test, as well as specific response tests to the Spike protein of interferon γ-producing T cells by flow cytometry from peripheral blood samples of participating individuals.

Unfortunately, during the study some of the participants acquired SARS-COV-2 infection, so the number of useful samples for immunological analysis was variable, but still with a number of individuals that was sufficient to have basic statistical conclusions. Notwithstanding the foregoing, none of the participants had serious adverse events that put their life or health at risk, nor adverse events of severe intensity, but only mild or moderate, so the analyzed dose is considered safe.

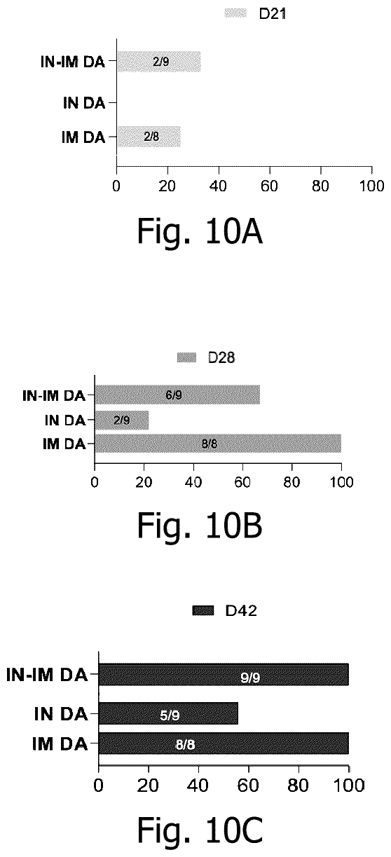

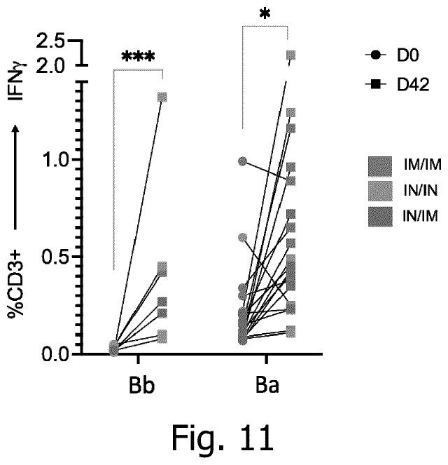

As can be seen in FIGS. 10A, 10B and 10C, although at day 21, after the first vaccination very few individuals had shown antibodies, by day 28, 7 days after the second vaccination, 100% of individuals in the group IM/IM were positive for neutralizing antibodies, and by day 42, 100% of individuals in the IN/IM and IM/IM groups were positive, and even in the IN/IN group 60% positivity was achieved, which suggests a delayed but equally effective effect of the IN route.

This is confirmed when analyzing the results of FIG. 11, in which it is also noted that when analyzing individuals with low basal levels of antibodies, a statistically significant cellular response was obtained with the high dose tested, but that this cellular response was also significant in individuals who had elevated basal levels of interferon γ-producing T cells, either due to previous infections with other coronaviruses or asymptomatic SARS-COV-2 some time ago.

Based on the examples, it is apparent that the use of an active Newcastle Disease viral vector with the S protein of the SARS-COV-2 virus is useful to generate increased humoral and cellular responses in individuals with previous immunity against the SARS-COV-2 virus, preferably in a high dose by the intramuscular or intranasal routes, and more preferably by the intranasal route.

Therefore, even though specific embodiments of the invention have been illustrated and described, it should be emphasized that numerous modifications to the invention are possible, such as the virus used as the viral vector, and the exogenous viral sequence used. Therefore, the present invention should not be considered as restricted except as required by the prior art and the appended claims.

1.-17. (canceled)

18. A coronavirus disease-19 (COVID-19) vaccine comprising
an active Newcastle disease virus comprising an exogenous nucleotide sequence encoding antigenic sites of severe acute respiratory syndrome coronavirus 2 (SARS-COV-2), and
a pharmaceutically acceptable vehicle and/or excipient,
wherein the COVID-19 vaccine does not comprise an adjuvant and is adapted to increase the percentage of interferon γ-producing T cells and neutralizing antibody titers in individuals with previous immunity to the SARS-COV-2.

19. The COVID-19 vaccine according to claim 18, wherein the vaccine is formulated for intranasal administration.

20. The COVID-19 vaccine according to claim 18, wherein the vaccine is formulated for intramuscular administration.

21. The COVID-19 vaccine according to claim 18, wherein the previous immunity was acquired by vaccination or a SARS-COV-2 infection.

22. The COVID-19 vaccine according to claim 18, wherein the previous immunity was acquired by vaccination with a messenger ribonucleic acid (mRNA) vaccine, a viral vector vaccine, or an inactivated SARS-COV-2 virus vaccine.

23. The vaccine against COVID-19 according to claim 18, wherein the previous immunity was acquired by COVID-19.

24. The COVID-19 vaccine according to claim 18, wherein the COVID-19 vaccine comprises at least 1×10^{8.0} active Newcastle disease virus particles measured by CEID_{50%}.

25. The COVID-19 vaccine according to claim 18, wherein the exogenous gene has a nucleotide sequence of a spike (S) protein that has at least 80% sequence identity with a nucleotide sequence encoding a S1 subunit and a S2 subunit of a spike (S) glycoprotein of SARS-COV-2 stabilized in its prefusion form by the inclusion of at least two proline substitutions in the S2 subunit.

26. The COVID-19 vaccine according to claim 25, wherein the exogenous gene has a nucleotide sequence that has at least 80% sequence identity with any sequence that translates into the amino acid sequence of SEQ ID NO:1.

27. A recombinant active Newcastle disease virus comprising an exogenous nucleotide sequence encoding antigenic sites of a severe acute respiratory syndrome coronavirus 2 (SARS-COV-2), wherein the recombinant active Newcastle disease virus is adapted to increase the percentage of interferon γ-producing T cells and neutralizing antibody titers in individuals with previous immunity to SARS-COV-2.

28. The recombinant active Newcastle disease virus according to claim 27, wherein the recombinant active Newcastle disease virus comprises at least 1×10^{8.0} recombinant active Newcastle disease virus particles measured by CEID_{50%}.

29. The recombinant active Newcastle disease virus according to claim 27, wherein the exogenous gene has a nucleotide sequence of a spike (S) protein that has at least 80% sequence identity with a nucleotide sequence encoding an S1 subunit and an S2 subunit of a spike (S) glycoprotein of SARS-COV-2 stabilized in its prefusion form by the inclusion of at least two proline substitutions in the S2 subunit.

30. The recombinant active Newcastle disease virus according to claim 29, wherein the exogenous gene has a nucleotide sequence has at least 80% sequence identity with any sequence that translates into the amino acid sequence of SEQ ID NO:1.

31. A method of enhancing a cellular and antibody response to severe acute respiratory syndrome coronavirus 2 (SARS-COV-2) in a subject, the method comprising administering the recombinant active Newcastle disease virus of claim 27 to a subject that has previous immunity to SARS-COV-2, wherein the recombinant active Newcastle disease virus increases the percentage of interferon γ-producing T cells and neutralizing antibody titers in the subject with previous immunity to SARS-COV-2.

32. The method of claim 31, wherein at least 1×10^{8.0} recombinant active Newcastle disease virus particles measured by CEID^{50%} is administered to the subject.

33. The method of claim 31, wherein the exogenous gene has a nucleotide sequence of a spike (S) protein that has at least 80% sequence identity with a nucleotide sequence encoding an S1 subunit and an S2 subunit of a spike (S) glycoprotein of SARS-COV-2 stabilized in its prefusion form by the inclusion of at least two proline substitutions in the S2 subunit.

34. The method of claim 31, wherein the exogenous gene has a nucleotide sequence has at least 80% sequence identity with any sequence that translates into the amino acid sequence of SEQ ID NO:1.

35. The method of claim 31, wherein the previous immunity was acquired by vaccination or a SARS-COV-2 infection.

36. The method of claim 31, wherein the previous immunity was acquired by vaccination with a messenger ribonucleic acid (mRNA) vaccine, a viral vector vaccine, or an inactivated SARS-COV-2 virus vaccine.

37. The method of claim 31, wherein the previous immunity was acquired by COVID-19.
